(19) United States
(12) Patent Application Publication
(10) Pub. No.: US 2019/0142388 A1
(43) Pub. Date: May 16, 2019

(54) METHODS AND APPARATUS FOR CONFIGURING AN ULTRASOUND DEVICE WITH IMAGING PARAMETER VALUES

(71) Applicants: Brenda Gonyeau, Feeding Hills, MA (US); Karl Thiele, Andover, MA (US); Nathan Silberman, Brooklyn, NY (US); Christophe Meyer, New York, NY (US)

(72) Inventors: Brenda Gonyeau, Feeding Hills, MA (US); Karl Thiele, Andover, MA (US); Nathan Silberman, Brooklyn, NY (US); Christophe Meyer, New York, NY (US)

(73) Assignee: Butterfly Network, Inc., Guilford, CT (US)

(21) Appl. No.: 16/192,620

(22) Filed: Nov. 15, 2018

Related U.S. Application Data
(60) Provisional application No. 62/586,469, filed on Nov. 15, 2017.

(51) Int. Cl. A61B 8/08; G16H 30/40; A61B 8/00

(52) U.S. Cl. CPC A61B 8/5207; G16H 30/40; G06T 7/0012; A61B 8/5215; A61B 8/48; A61B 8/58

(57) ABSTRACT

Aspects of the technology described herein relate to configuring an ultrasound device with ultrasound imaging presets. Some embodiments include configuring an ultrasound device with different sets of imaging parameter values without receiving user input regarding configuration of the ultrasound device between configuration of the ultrasound device with one set of imaging parameter values and configuration of the ultrasound device with another set of imaging parameter values. Some embodiments include automatically configuring an ultrasound device with a set of imaging parameter values based on automatically determining an anatomical location being imaged by the ultrasound device. Some embodiments include automatically configuring an ultrasound device with a cardiac preset when a cardiac region is being imaged and automatically configuring the ultrasound device with an abdominal preset when an abdominal region is being imaged during a FAST or eFAST exam.

### CROSS-REFERENCE TO RELATED APPLICATIONS

This application claims the benefit under 35 U.S.C. § 119(e) of U.S. Provisional Patent Application Ser. No. 62/586,469, filed Nov. 15, 2017 under Attorney Docket No. B1348.70066US00, and entitled “METHODS AND APPARATUS FOR CONFIGURING AN ULTRASOUND DEVICE BASED ON ULTRASOUND IMAGING PRESETS,” which is hereby incorporated herein by reference in its entirety.

### FIELD

Generally, the aspects of the technology described herein relate to ultrasound data collection. Some aspects relate to configuring an ultrasound device with imaging parameter values.

### BACKGROUND

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using a probe), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, a method includes configuring, with a processing device in operative communication with an ultrasound device, the ultrasound device with different sets of imaging parameter values without intervening imaging parameter configuration user input.

In some embodiments, configuring the ultrasound device with the different sets of imaging parameter values is based at least in part on determining that the ultrasound device has changed from imaging a first anatomical location to imaging a second anatomical location. In some embodiments, determining that the ultrasound device has changed from imaging the first anatomical location to imaging the second anatomical location is performed by analyzing ultrasound data collected by the ultrasound device or by analyzing at least one ultrasound image generated based on the ultrasound data collected by the ultrasound device. In some embodiments, determining that the ultrasound device has changed from imaging the first anatomical location to imaging the second anatomical location is performed by analyzing an optical image or an optical video obtained with a camera on the processing device. In some embodiments, determining that the ultrasound device has changed from imaging the first anatomical location to imaging the second anatomical location is performed by analyzing accelerometer data from the ultrasound device. In some embodiments, determining that the ultrasound device has changed from imaging the first anatomical location to imaging the second anatomical location includes using at least one statistical model. In some embodiments, configuring the ultrasound device with the different sets of imaging parameter values is performed subsequent to and based on receiving a user selection of a FAST or eFAST exam preset.

According to another aspect, a method includes automatically configuring, with a processing device in operative communication with an ultrasound device, the ultrasound device with a set of imaging parameter values based on automatically determining an anatomical location being imaged by the ultrasound device. In some embodiments, determining the anatomical location being imaged by the ultrasound device includes analyzing ultrasound data collected by the ultrasound device or analyzing at least one ultrasound image generated based on the ultrasound data collected by the ultrasound device. In some embodiments, determining the anatomical location being imaged by the ultrasound device includes analyzing an optical image or an optical video obtained with a camera on the processing device. In some embodiments, determining the anatomical location being imaged by the ultrasound device includes analyzing accelerometer data from the ultrasound device. In some embodiments, determining the anatomical location being imaged by the ultrasound device includes using at least one statistical model. In some embodiments, automatically configuring the ultrasound device with the set of imaging parameter values based on automatically determining the anatomical location being imaged by the ultrasound device is performed subsequent to and based on receiving a user selection of a FAST or eFAST exam preset. In some embodiments, automatically configuring the ultrasound device with the set of imaging parameter values based on automatically determining the anatomical location being imaged by the ultrasound device is performed without receiving user input regarding configuration of the ultrasound device. In some embodiments, the set of imaging parameter values corresponds to the anatomical location being imaged by the ultrasound device.

According to another aspect, a method of operating an ultrasound device for a FAST or eFAST exam includes automatically configuring, with a processing device in operative communication with the ultrasound device, the ultrasound device with a cardiac preset when a cardiac region is being imaged and automatically configuring the ultrasound device with an abdominal preset when an abdominal region is being imaged during the FAST or eFAST exam.

In some embodiments, the method further includes automatically determining whether the cardiac region or the abdominal region is being imaged by the ultrasound device. In some embodiments, determining whether the cardiac region or the abdominal region is being imaged by the ultrasound device includes analyzing ultrasound data collected by the ultrasound device or analyzing at least one ultrasound image generated based on the ultrasound data collected by the ultrasound device. In some embodiments, determining whether the cardiac region or the abdominal region is being imaged by the ultrasound device includes analyzing an optical image or an optical video obtained with a camera on the processing device. In some embodiments, determining whether the cardiac region or the abdominal region is being imaged by the ultrasound device includes analyzing accelerometer data from the ultrasound device. In some embodiments, determining whether the cardiac region or the abdominal region is being imaged by the ultrasound device includes using at least one statistical model. In some embodiments, automatically configuring the ultrasound device with a cardiac preset when a cardiac region is being imaged and automatically configuring the ultrasound device with an abdominal preset when an abdominal region is being imaged during the FAST or eFAST exam is performed without receiving user input regarding configuration of the ultrasound device.

Some aspects include at least one non-transitory computer-readable storage medium storing processor-executable instructions that, when executed by at least one processor, cause the at least one processor to perform the above aspects and embodiments. Some aspects include an apparatus having a processing device configured to perform the above aspects and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 shows an example process for configuring an ultrasound device with imaging parameter values in accordance with certain embodiments described herein;

FIG. 2 shows a schematic block diagram illustrating aspects of an example ultrasound system upon which various aspects of the technology described herein may be practiced;

FIG. 3 is a schematic block diagram illustrating aspects of another example ultrasound system upon which various aspects of the technology described herein may be practiced;

FIG. 4 shows an example convolutional neural network that is configured to analyze an image; and

FIG. 5 shows an example of a “virtuous circle” for continuously improving the performance of certain processes and systems described herein.

### DETAILED DESCRIPTION

Fast assessment with sonography in trauma (FAST) exams are exams performed using ultrasound imaging to rapidly screen for blood around a subject's heart and abdominal organs following trauma. FAST exams may be performed, for example, by emergency medicine physicians or paramedics. In a FAST exam, ultrasound imaging is used to look for free fluid in four anatomical locations in the abdomen and heart: the perihepatic space, the perisplenic space, the pericardium, and the pelvis.

An ultrasound imaging system may include preprogrammed image parameter values for configuring an ultrasound device to image various anatomical locations. For example, a given anatomical location may be located at a certain depth from the surface of a subject, and the depth may determine imaging parameters such as frequency and frame rate. One or more imaging parameter values may be preprogrammed into a processing device in communication with an ultrasound device, and may be referred to as a preset. Different anatomical locations may have different presets. Thus, for example, a user wishing to scan a subject's heart may select a cardiac preset on the ultrasound imaging system, and this selection may cause the ultrasound imaging system to configure the ultrasound device with the preprogrammed imaging parameter values for cardiac ultrasound imaging. In some embodiments, information used to generate the imaging parameter values of a preset may be preprogrammed into the processing device.

Even though FAST exams image portions of both the heart and the abdomen, a FAST exam will typically only use a single general FAST preset in order to reduce the setup time a user must perform to initiate such a rapid exam. On the other hand, a single general FAST preset may be considered “compromised” from an imaging perspective in that the preset may not be optimized for imaging any one anatomical location imaged during a FAST exam. For example, a cardiac preset may use imaging parameter values for imaging at a depth of 13 cm, an abdominal preset may use imaging parameter values for imaging at a depth of 16 cm, and a general FAST preset may use imaging parameter values for imaging at a depth of 18 cm. Accordingly, ultrasound images of the heart and abdomen resulting from a general FAST exam may be lower in quality and clinical value than ultrasound images of the heart and abdomen collected using presets specifically optimized for the heart and abdomen, respectively.

Extended FAST (eFAST) exams image the same anatomical locations as a FAST exam, in addition to imaging the lungs with bilateral anterior thoracic imaging. In a similar vein as above, because a single eFAST preset is typically used, ultrasound images of the heart, abdomen, and lungs resulting from an eFAST exam may be lower in quality and clinical value than ultrasound images of the heart, abdomen, and lungs collected using parameter values specifically optimized for the heart and abdomen, respectively.

The inventors have recognized that it may be possible to improve the quality and clinical value of ultrasound images collected during FAST and eFAST exams by automatically determining the anatomical location being imaged during a FAST or eFAST exam and selecting a preset corresponding to the anatomical location being imaged. Thus, for example, a user may select a single eFAST ultrasound imaging preset on the ultrasound imaging system, and begin the eFAST exam by imaging the abdomen. The ultrasound imaging system may automatically determine that the abdomen is being imaged and select an abdominal preset for use during the imaging. The user may continue to image the abdomen using the abdominal preset, and then move to image the heart. The ultrasound imaging system may automatically determine that now the heart is being imaged and change from using the abdominal preset to using a cardiac preset during the imaging. The user may continue to image the heart using the cardiac preset, and then move to image the lungs. The ultrasound imaging system may automatically determine that now the lungs are being imaged and change from using the cardiac preset to using a lung preset during the imaging. The user may continue to image the lungs using the lung preset. As another example, a user may select a single eFAST ultrasound imaging preset on the ultrasound imaging system, and begin the eFAST exam by imaging the lungs. The ultrasound imaging system may automatically determine that the lungs are being imaged and select a lung preset for use during the imaging. The user may continue to image the lungs using the lung preset, and then move to image the heart. The ultrasound imaging system may automatically determine that the heart is being imaged and select a cardiac preset for use during the imaging. The user may continue to image the heart using the cardiac preset, and then move to image the abdomen. The ultrasound imaging system may automatically determine that now the abdomen is being imaged and change from using the cardiac preset to using an abdominal preset for use during the imaging. The user may continue to image the abdomen using the abdominal preset. Therefore, ultrasound images of the abdomen may be collected using an abdominal preset and ultrasound images of the heart may be collected using a cardiac preset.

To determine which anatomical location is being imaged during an imaging session, the ultrasound imaging system may analyze ultrasound data/ultrasound images currently being collected, optical images or video depicting the ultrasound device and the subject, and/or accelerometer data from the ultrasound device. The ultrasound imaging system may use deep learning techniques for performing the analysis.

The embodiments discussed herein are not limited to FAST/eFAST exams and FAST/eFAST presets, but may be used in any type of imaging exam and any presets. For example, a user may select a gallbladder preset and proceed to image the gallbladder, but then move to imaging the carotid artery. The ultrasound imaging system may automatically determine that now the lungs are being imaged and change from using the gallbladder preset to using a vascular preset. In some embodiments, the user need not select any preset, as a default preset may be implemented initially.

The solution discussed above is unconventional because selection of an ultrasound imaging preset (e.g., a FAST or eFAST exam preset) prior to an ultrasound imaging session typically results in configuration of an ultrasound device using a single set of imaging parameter values for the duration of the ultrasound imaging session, or until another ultrasound imaging preset is selected. In contrast, in the solution discussed above, selection of a preset may trigger the ultrasound imaging system to use multiple sets of imaging parameter values from multiple presets over the course of a single ultrasound session (e.g., a FAST or eFAST exam). In the specific cases of FAST and eFAST exams, the solution discussed above is also unconventional because the use of a single preset during a FAST or eFAST exam is typically considered beneficial, in that it may help to reduce the setup time a user must perform to initiate a FAST or eFAST exam, which is intended to be a rapid exam. In contrast, the solution discussed above may use multiple presets in order to improve the quality of collected ultrasound images, but without increasing setup time because the selection of presets is automatic.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

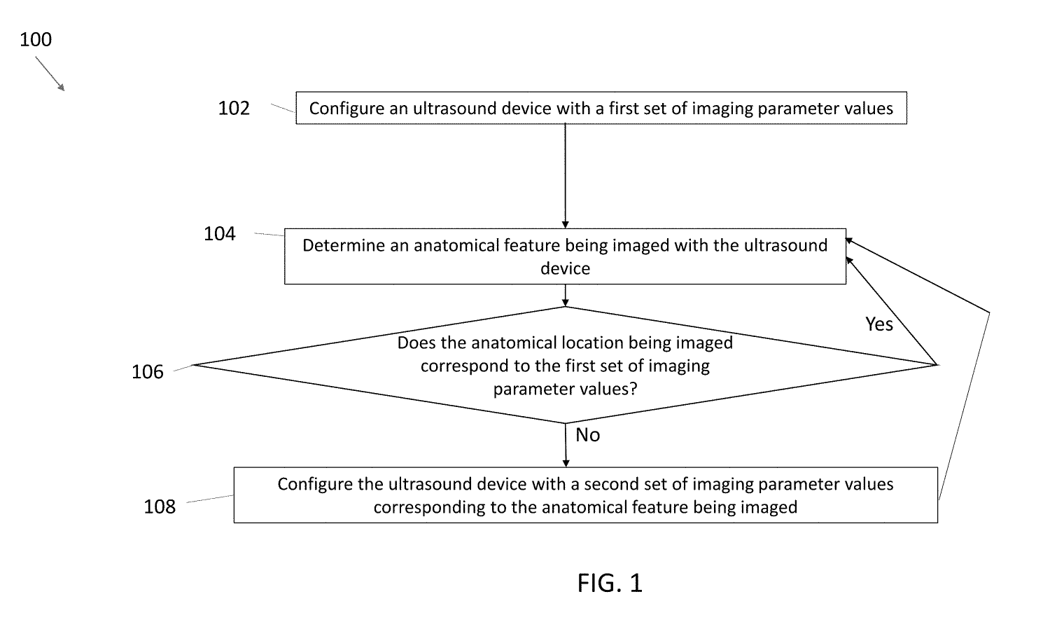

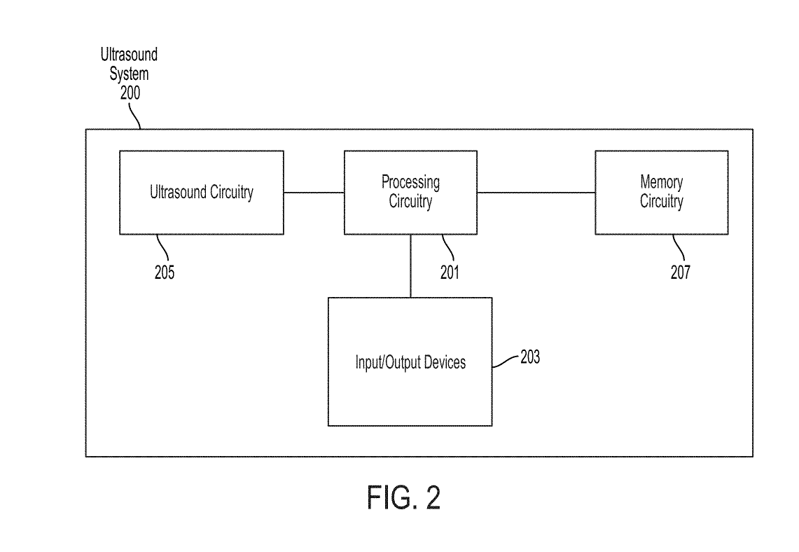

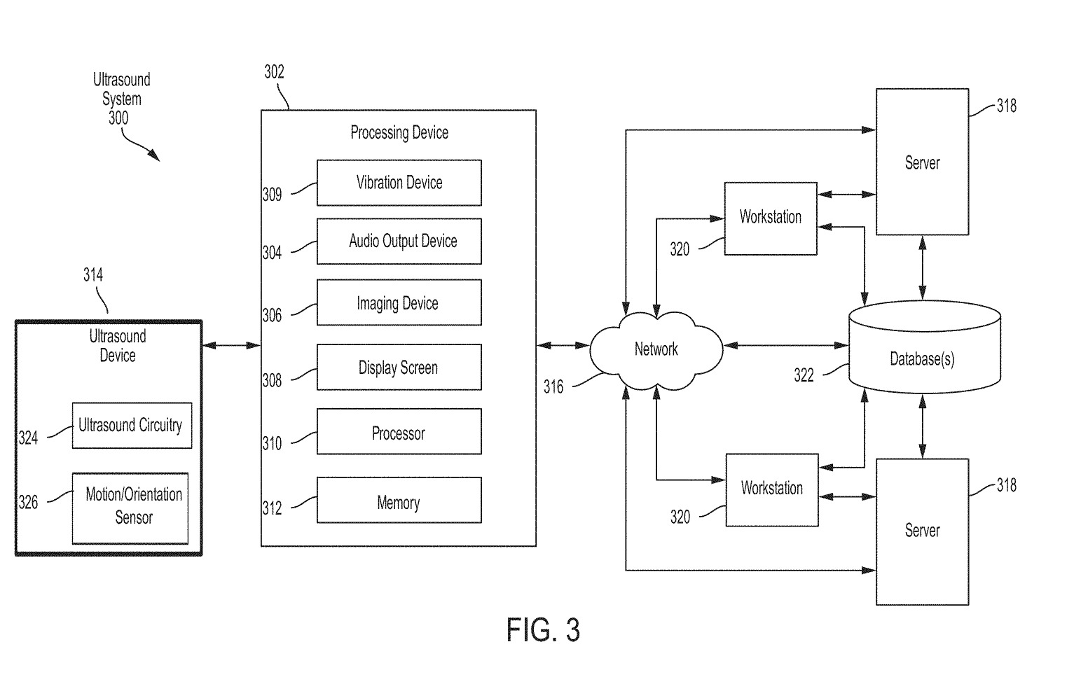

FIG. 1 shows an example process 100 for configuring an ultrasound device with imaging parameter values in accordance with certain embodiments described herein. The process 100 is performed by a processing device that is part of or in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). Ultrasound devices and processing devices are discussed in more detail with reference to FIGS. 2-3.

In act 102, the processing device configures the ultrasound device with a first set of imaging parameter values. For example, the processing device may transmit an instruction/instructions to the ultrasound device to trigger configuration of the ultrasound device with the first imaging parameter values. In some embodiments, the first set of imaging parameter values may be a general FAST or eFAST exam preset. In some embodiments, the first set of imaging parameter values may be a preset for a specific anatomical location, such as an abdominal preset or a cardiac preset. In some embodiments, the first set of imaging parameter values may be a default preset that is automatically selected at the beginning of the process 100. In some embodiments, prior to act 102, the user may have selected an anatomical location or imaging protocol from a menu of preset options, and the first set of imaging parameter values may correspond to the selected anatomical location or imaging protocol. For example, if the FAST preset is selected, the first set of imaging parameter values may be a general FAST exam preset. If the cardiac preset is selected, the first set of imaging parameter values may be a cardiac preset. If the abdominal preset is selected, the first set of imaging parameter values may correspond to an abdominal preset. The process 100 proceeds from act 102 to act 104.

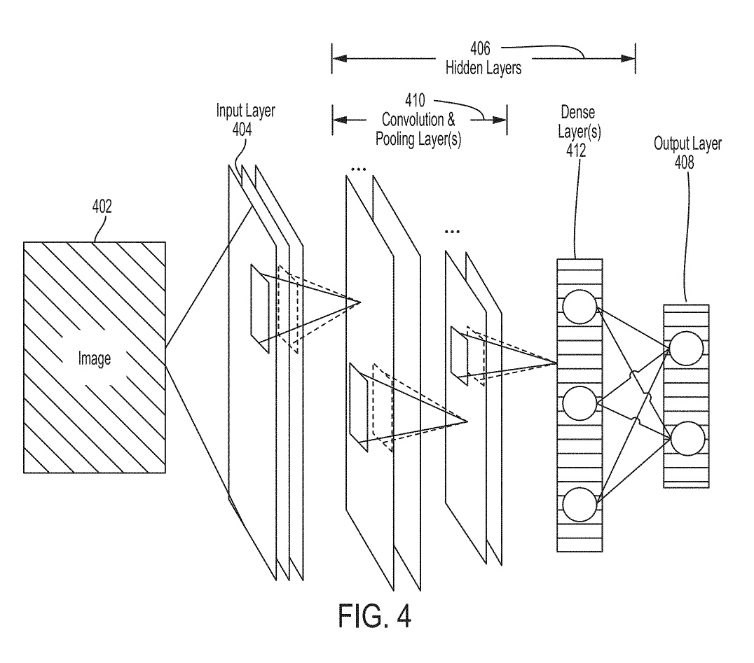

In act 104, the processing device determines an anatomical location being imaged with the ultrasound device. In some embodiments, to determine the anatomical location being imaged, the processing device may analyze ultrasound data (e.g., raw acoustical data, scan lines, or one or more ultrasound images) received from the ultrasound device, or data (e.g., scan lines or one or more ultrasound images) generated from the ultrasound data received from the ultrasound device. In some embodiments, analyzing the ultrasound data may involve using one or more statistical models and/or deep learning techniques. The statistical models may include a convolutional neural network, a fully connected neural network, a recurrent neural network (e.g., a long short-term memory (LSTM) recurrent neural network), a random forest, a support vector machine, a linear classifier, and/or any other statistical model. The statistical models may be trained to determine, based on the ultrasound data, an anatomical location that was being imaged when the ultrasound data was collected. The statistical models may be trained on multiple sets of ultrasound data each collected from a respective anatomical location and labeled with the respective anatomical location. Statistical models and deep learning techniques are discussed in more detail with reference to FIG. 4.

In some embodiments, to determine the anatomical location being imaged, the processing device may analyze data such as one or more optical images or an optical video received from the camera of the processing device (e.g., a camera on a smartphone). For example, the user may hold the ultrasound device on the subject with one hand and hold the processing device in the other hand such that the ultrasound device and the subject are in view of the camera of the processing device. In some embodiments, analyzing the optical images or video may involve using one or more statistical models and/or deep learning techniques. The statistical models may be trained to determine, based on one or more optical images or an optical video of an ultrasound device on a subject, on which anatomical location or region of the subject (e.g., abdomen, wrist, carotid artery, or legs) the ultrasound device is located. The statistical models may be trained on multiple sets of optical images or video each depicting an ultrasound device on a respective anatomical location and labeled with the respective anatomical location.

In some embodiments, to determine the anatomical location being imaged, the processing device may analyze data collected from an accelerometer on the ultrasound device. Assuming a particular orientation of the subject relative to gravity, the orientation of the ultrasound device relative to gravity may be different depending on the anatomical location being imaged, and the data from the accelerometer may therefore be different. For example, if the subject is supine, the ultrasound device may be oriented perpendicular to gravity if the ultrasound device is imaging the liver, kidneys, or the apical four-chamber view of the heart. The ultrasound device may be oriented parallel to gravity if the ultrasound device is imaging the aorta, inferior vena cava, bladder, lung, or the parasternal long axis view of the heart. Thus, the processing device may use the accelerometer data from the ultrasound device to determine the anatomical location being imaged, or at least narrow down the options of anatomical locations being imaged.

In some embodiments, the processing device may use more than one method for determining the anatomical location being imaged by the ultrasound device. For example, the processing device may use optical images or video to determine a general anatomical region being imaged, and then use accelerometer data to further narrow down the options for the anatomical location being imaged within the anatomical region.

In some embodiments, the processing device may itself determine the anatomical location being imaged. In some embodiments, the processing device may upload ultrasound data, optical images or video, and/or accelerometer data to a remote server that determines the anatomical location being imaged, and the processing device may download the result of the determination from the remote server.

In act 106, the processing device determines whether the anatomical location being imaged corresponds to the first set of imaging parameter values (i.e., the set of imaging parameter values which the ultrasound device is currently configured). For example, if in act 106 the processing device determines that the abdomen is being imaged, the processing device may determine whether the first set of imaging parameter values is an abdominal preset. As another example, if in act 106 the processing device determines that the heart is currently imaged, the processing device may determine whether the first set of imaging parameter values is a cardiac preset. In some embodiments, in act 106, the options for anatomical locations that the processing device can determine as being imaged may only correspond to one of the available presets on the ultrasound device or none of the available presets. If statistical models are used to determine the anatomical location being imaged, the output of the statistical models may only correspond to one of the available presets or none of the available presets. For example, if the available presets are an abdominal preset and a cardiac preset, the processing device may only determine in act 106 that the abdomen is being imaged, the heart is being imaged, or neither the abdomen nor the heart is being imaged. In some embodiments, the processing device may access a data structure of associations between options for anatomical locations that the processing device can determine as being imaged and the available presets. The preset associated with an anatomical location may be the available preset that corresponds to, subsumes, or is most relevant to the anatomical location being imaged. For example, if the processing device determines in act 106 that the liver is being imaged, the processing device may determine that the abdominal preset is associated with the liver.

If, at act 106, the processing device determines that the anatomical location being imaged corresponds to the first set of imaging parameter values, the process 100 may return from act 106 to act 104, in which the processing device may continue to determine the anatomical location being imaged as the ultrasound imaging continues. If the processing device determines that the anatomical location being imaged does not correspond to the first set of imaging parameter values, the process 100 may proceed from act 106 to act 108. In some embodiments, the process 100 may proceed from act 106 to act 108 only if the anatomical location being imaged does not correspond to the first set of imaging parameter values and only if the anatomical location being imaged corresponds to an available preset. If the anatomical location being imaged does not correspond to the first set of imaging parameter values and the anatomical location being imaged does not correspond to an available preset, or cannot be determined, the process 100 may return from act 106 to act 104.

In act 108, the processing device configures the ultrasound device with a second set of imaging parameter values corresponding to the anatomical location being imaged with the ultrasound device. For example, if the anatomical location being imaged is the heart, and the first set of imaging parameter values is an abdominal preset, the processing device may configure the ultrasound device with a cardiac preset, thereby undoing configuration of the ultrasound device with the abdominal preset. The processing device may determine the anatomical location being imaged continuously or periodically throughout the duration of the ultrasound imaging, and the processing device may also continuously or periodically update the set of imaging parameter values with which the ultrasound device is configured. For example, an eFAST exam may involve imaging portions of the abdomen, then imaging portions of the heart, and then imaging portions of the lung. Accordingly, the processing device may initially determine that the abdomen is being imaged and configure the ultrasound device with an abdominal preset. The processing device may subsequently determine that the heart is being imaged and configure the ultrasound device with a cardiac preset. The processing device may subsequently determine that the lungs are being imaged and configure the ultrasound device with a lung preset. In some cases, more than three presets may be used to configure the ultrasound device during a single eFAST exam, for example, presets corresponding to the perihepatic space, the perisplenic space, the pericardium, the pelvis, and/or the lungs. As another example, a FAST exam may involve imaging portions of the abdomen and then imaging portions of the heart. Accordingly, the processing device may initially determine that the abdomen is being imaged and configure the ultrasound device with an abdominal preset, and the processing device may subsequently determine that the heart is being imaged and configure the ultrasound device with a cardiac preset. In some cases, more than two presets may be used to configure the ultrasound device during a single FAST exam. For example, presets corresponding to the perihepatic space, the perisplenic space, the pericardium, and/or the pelvis may be implemented during a single FAST exam. If the ultrasound imaging terminates after act 108 (e.g., the user shuts off the ultrasound device or manually selects another preset), the process 100 may terminate. Otherwise, the process 100 may return from act 108 to act 104.

It should be appreciated that the processing may change the configuration of the ultrasound device from the first set of imaging parameter values to the second set of imaging parameter values without intervening imaging parameter configuration user input. In other words, the processing device may not receive any user input regarding configuration of the ultrasound device in between when the ultrasound device is configured with the first set of imaging parameter values and when the ultrasound device is configured with the second set of imaging parameter values. For example, the user may not need to select a different preset to cause the processing device to change the configuration of the ultrasound device.

In some embodiments, the length of time required to determine the anatomical location being imaged (at act 104) and to configure the ultrasound device with a preset corresponding to the anatomical location being imaged (at act 108) may correspond to 1-2 frames of ultrasound imaging.

In some embodiments, acts 104, 106, and 108 may be performed automatically. For example, acts 104, 106, and 108 may occur automatically subsequent to and based on a selection of an option corresponding to a FAST or eFAST exam (e.g., selection of a FAST or eFAST exam preset from a menu of preset options). In other words, selection of the FAST or eFAST exam preset may cause the processing device to automatically configure the ultrasound device with the second set of imaging parameter values based on the determination of the anatomical feature being imaged. In such embodiments, selection of a different preset may not trigger acts 104, 106, and 108 to occur. In other words, selection of a different preset may cause the processing device to configure the ultrasound device with the selected preset's imaging parameter values for the duration of the imaging session (or until a different preset is selected), regardless of the anatomical location being imaged. In some embodiments, acts 104, 106, and 108 may be triggered regardless of the option selected prior to act 104. For example, a user may select a gallbladder preset and proceed to image the gallbladder, but then move to imaging the lungs. The ultrasound imaging system may automatically determine at act 104 that now the lungs are being imaged and automatically change from using the gallbladder preset to using the lungs preset at act 108.

While the above description has described the process 100 as being performed by a processing device in operative communication with an ultrasound device, in some embodiments, the ultrasound device itself may perform the process 100.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

FIG. 2 shows a schematic block diagram illustrating aspects of an example ultrasound system 200 upon which various aspects of the technology described herein may be practiced. For example, one or more components of the ultrasound system 200 may perform any of the processes described herein. As shown, the ultrasound system 200 includes processing circuitry 201, input/output devices 203, ultrasound circuitry 205, and memory circuitry 207.

The ultrasound circuitry 205 may be configured to generate ultrasound data that may be employed to generate an ultrasound image. The ultrasound circuitry 205 may include one or more ultrasonic transducers monolithically integrated onto a single semiconductor die. The ultrasonic transducers may include, for example, one or more capacitive micromachined ultrasonic transducers (CMUTs), one or more CMOS ultrasonic transducers (CUTs), one or more piezoelectric micromachined ultrasonic transducers (PMUTs), and/or one or more other suitable ultrasonic transducer cells. In some embodiments, the ultrasonic transducers may be formed on the same chip as other electronic components in the ultrasound circuitry 205 (e.g., transmit circuitry, receive circuitry, control circuitry, power management circuitry, and processing circuitry) to form a monolithic ultrasound device.

The processing circuitry 201 may be configured to perform any of the functionality described herein. The processing circuitry 201 may include one or more processors (e.g., computer hardware processors). To perform one or more functions, the processing circuitry 201 may execute one or more processor-executable instructions stored in the memory circuitry 207. The memory circuitry 207 may be used for storing programs and data during operation of the ultrasound system 200. The memory circuitry 207 may include one or more storage devices such as non-transitory computer-readable storage media. The processing circuitry 201 may control writing data to and reading data from the memory circuitry 207 in any suitable manner.

In some embodiments, the processing circuitry 201 may include specially-programmed and/or special-purpose hardware such as an application-specific integrated circuit (ASIC). For example, the processing circuitry 201 may include one or more graphics processing units (GPUs) one or more tensor processing units (TPUs). TPUs may be ASICs specifically designed for machine learning (e.g., deep learning). The TPUs may be employed to, for example, accelerate the inference phase of a neural network.

The input/output (I/O) devices 203 may be configured to facilitate communication with other systems and/or an operator. Example I/O devices 203 that may facilitate communication with an operator include: a keyboard, a mouse, a trackball, a microphone, a touch screen, a printing device, a display screen, a speaker, and a vibration device. Example I/O devices 203 that may facilitate communication with other systems include wired and/or wireless communication circuitry such as BLUETOOTH, ZIGBEE, WiFi, and/or USB communication circuitry.

It should be appreciated that the ultrasound system 200 may be implemented using any number of devices. For example, the components of the ultrasound system 200 may be integrated into a single device. In another example, the ultrasound circuitry 205 may be integrated into an ultrasound device that is communicatively coupled with a processing device that includes the processing circuitry 201, the input/output devices 203, and the memory circuitry 207.

FIG. 3 is a schematic block diagram illustrating aspects of another example ultrasound system 300 upon which various aspects of the technology described herein may be practiced. For example, one or more components of the ultrasound system 300 may perform any of the processes described herein. As shown, the ultrasound system 300 includes an ultrasound device 314 in wired and/or wireless communication with a processing device 302. The ultrasound device 314 includes ultrasound circuitry 324 and a motion/orientation sensor 326. The processing device 302 includes an audio output device 304, an imaging device 306, a display screen 308, a processor 310, a memory 312, and a vibration device 309. The processing device 302 may communicate with one or more external devices over a network 316. For example, the processing device 302 may communicate with one or more workstations 320, servers 318, and/or databases 322.

The ultrasound device 314 may be configured to generate ultrasound data that may be employed to generate an ultrasound image. The ultrasound device 314 may be constructed in any of a variety of ways. In some embodiments, the ultrasound device 314 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 324 may be configured to generate the ultrasound data. The ultrasound circuitry 324 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 324 (e.g., transmit circuitry, receive circuitry, control circuitry, power management circuitry, and processing circuitry) to form a monolithic ultrasound device. The ultrasound device 314 may transmit ultrasound data and/or ultrasound images to the processing device 302 over a wired (e.g., through a lightning connector or a mini-USB connector) and/or wireless (e.g., using BLUETOOTH, ZIGBEE, and/or WiFi wireless protocols) communication link.

The motion and/or orientation sensor 326 may be configured to generate motion and/or orientation data regarding the ultrasound device 314. For example, the motion and/or orientation sensor 326 may be configured to generate to generate data regarding acceleration of the ultrasound device 314, data regarding angular velocity of the ultrasound device 314, and/or data regarding magnetic force acting on the ultrasound device 314 (which, due to the magnetic field of the earth, may be indicative of orientation relative to the earth). The motion and/or orientation sensor 326 may include an accelerometer, a gyroscope, and/or a magnetometer. Depending on the sensors present in the motion and/or orientation sensor 326, the motion and/or orientation data generated by the motion and/or orientation sensor 326 may describe three degrees of freedom, six degrees of freedom, or nine degrees of freedom for the ultrasound device 314. For example, the motion and/or orientation sensor may include an accelerometer, a gyroscope, and/or magnetometer. Each of these types of sensors may describe three degrees of freedom. If the motion and/or orientation sensor includes one of these sensors, the motion and/or orientation sensor may describe three degrees of freedom. If the motion and/or orientation sensor includes two of these sensors, the motion and/or orientation sensor may describe two degrees of freedom. If the motion and/or orientation sensor includes three of these sensors, the motion and/or orientation sensor may describe nine degrees of freedom. The ultrasound device 314 may transmit motion and/or orientation data to the processing device 302 over a wired (e.g., through a lightning connector or a mini-USB connector) and/or wireless (e.g., using BLUETOOTH, ZIGBEE, and/or WiFi wireless protocols) communication link.

Referring now to the processing device 302, the processing device 302 may be configured to process the ultrasound data from the ultrasound device 314 to generate ultrasound images for display on the display screen 308. The processing may be performed by, for example, the processor 310. The processor 310 may also be adapted to control the acquisition of ultrasound data with the ultrasound device 314. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

Additionally (or alternatively), the processing device 302 may be configured to perform any of the processes described herein (e.g., using the processor 310). For example, the processing device 302 may be configured to automatically determine an anatomical location being imaged and automatically select, based on the anatomical location being imaged, an ultrasound imaging preset corresponding to the anatomical location. As shown, the processing device 302 may include one or more elements that may be used during the performance of such processes. For example, the processing device 302 may include one or more processors 310 (e.g., computer hardware processors) and one or more articles of manufacture that include non-transitory computer-readable storage media such as the memory 312. The processor 310 may control writing data to and reading data from the memory 312 in any suitable manner. To perform any of the functionality described herein, the processor 310 may execute one or more processor-executable instructions stored in one or more non-transitory computer-readable storage media (e.g., the memory 312), which may serve as non-transitory computer-readable storage media storing processor-executable instructions for execution by the processor 310.

In some embodiments, the processing device 302 may include one or more input and/or output devices such as the audio output device 304, the imaging device 306, the display screen 308, and the vibration device 309. The audio output device 304 may be a device that is configured to emit audible sound such as a speaker. The imaging device 306 may be configured to detect light (e.g., visible light) to form an image such as a camera. The display screen 308 may be configured to display images and/or videos such as a liquid crystal display (LCD), a plasma display, and/or an organic light emitting diode (OLED) display. The vibration device 309 may be configured to vibrate one or more components of the processing device 302 to provide tactile feedback. These input and/or output devices may be communicatively coupled to the processor 310 and/or under the control of the processor 310. The processor 310 may control these devices in accordance with a process being executed by the process 310 (such as the process shown in FIG. 1). Similarly, the processor 310 may control the audio output device 304 to issue audible instructions and/or control the vibration device 309 to change an intensity of tactile feedback (e.g., vibration) to issue tactile instructions. Additionally (or alternatively), the processor 310 may control the imaging device 306 to capture non-acoustic images of the ultrasound device 314 being used on a subject to provide an operator of the ultrasound device 314 an augmented reality interface.

It should be appreciated that the processing device 302 may be implemented in any of a variety of ways. For example, the processing device 302 may be implemented as a handheld device such as a mobile smartphone or a tablet. Thereby, an operator of the ultrasound device 314 may be able to operate the ultrasound device 314 with one hand and hold the processing device 302 with another hand. In other examples, the processing device 302 may be implemented as a portable device that is not a handheld device such as a laptop. In yet other examples, the processing device 302 may be implemented as a stationary device such as a desktop computer.

In some embodiments, the processing device 302 may communicate with one or more external devices via the network 316. The processing device 302 may be connected to the network 316 over a wired connection (e.g., via an Ethernet cable) and/or a wireless connection (e.g., over a WiFi network). As shown in FIG. 3, these external devices may include servers 318, workstations 320, and/or databases 322. The processing device 302 may communicate with these devices to, for example, off-load computationally intensive tasks. For example, the processing device 302 may send an ultrasound image over the network 316 to the server 318 for analysis (e.g., to identify an anatomical location in the ultrasound) and receive the results of the analysis from the server 318. Additionally (or alternatively), the processing device 302 may communicate with these devices to access information that is not available locally and/or update a central information repository. For example, the processing device 302 may access the medical records of a subject being imaged with the ultrasound device 314 from a file stored in the database 322. In this example, the processing device 302 may also provide one or more captured ultrasound images of the subject to the database 322 to add to the medical record of the subject. For further discussion of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 and published as U.S. Pat. Publication No. 2017/0360397 A1 (and assigned to the assignee of the instant application), which is incorporated by reference herein in its entirety.

Aspects of the technology described herein relate to the application of automated image processing techniques to analyze images, such as ultrasound images. In some embodiments, the automated image processing techniques may include machine learning techniques such as deep learning techniques. Machine learning techniques may include techniques that seek to identify patterns in a set of data points and use the identified patterns to make predictions for new data points. These machine learning techniques may involve training (and/or building) a model using a training data set to make such predictions. The trained model may be used as, for example, a classifier that is configured to receive a data point as an input and provide an indication of a class to which the data point likely belongs as an output.

Deep learning techniques may include those machine learning techniques that employ neural networks to make predictions. Neural networks typically include a collection of neural units (referred to as neurons) that each may be configured to receive one or more inputs and provide an output that is a function of the input. For example, the neuron may sum the inputs and apply a transfer function (sometimes referred to as an “activation function”) to the summed inputs to generate the output. The neuron may apply a weight to each input, for example, to weight some inputs higher than others. Example transfer functions that may be employed include step functions, piecewise linear functions, and sigmoid functions. These neurons may be organized into a plurality of sequential layers that each include one or more neurons. The plurality of sequential layers may include an input layer that receives the input data for the neural network, an output layer that provides the output data for the neural network, and one or more hidden layers connected between the input and output layers. Each neuron in a hidden layer may receive inputs from one or more neurons in a previous layer (such as the input layer) and provide an output to one or more neurons in a subsequent layer (such as an output layer).

A neural network may be trained using, for example, labeled training data. The labeled training data may include a set of example inputs and an answer associated with each input. For example, the training data may include a plurality of ultrasound images that are each labeled with an anatomical location that is contained in the respective ultrasound image. In this example, the ultrasound images may be provided to the neural network to obtain outputs that may be compared with the labels associated with each of the ultrasound images. One or more characteristics of the neural network (such as the interconnections between neurons (referred to as edges) in different layers and/or the weights associated with the edges) may be adjusted until the neural network correctly classifies most (or all) of the input images.

Once the training data has been created, the training data may be loaded to a database (e.g., an image database) and used to train a neural network using deep learning techniques. Once the neural network has been trained, the trained neural network may be deployed to one or more processing devices. It should be appreciated that the neural network may be trained with any number of sample patient images. For example, a neural network may be trained with as few as 7 or so sample patient images, although it will be appreciated that the more sample images used, the more robust the trained model data may be.

In some applications, a neural network may be implemented using one or more convolution layers to form a convolutional neural network. An example convolutional neural network is shown in FIG. 4 that is configured to analyze an image 402. As shown, the convolutional neural network includes an input layer 404 to receive the image 402, an output layer 408 to provide the output, and a plurality of hidden layers 406 connected between the input layer 404 and the output layer 408. The plurality of hidden layers 406 includes convolution and pooling layers 410 and dense layers 412.

The input layer 404 may receive the input to the convolutional neural network. As shown in FIG. 4, the input the convolutional neural network may be the image 402. The image 402 may be, for example, an ultrasound image.

The input layer 404 may be followed by one or more convolution and pooling layers 410. A convolutional layer may include a set of filters that are spatially smaller (e.g., have a smaller width and/or height) than the input to the convolutional layer (e.g., the image 402). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 410 may be followed by dense layers 412. The dense layers 412 may include one or more layers each with one or more neurons that receives an input from a previous layer (e.g., a convolutional or pooling layer) and provides an output to a subsequent layer (e.g., the output layer 408). The dense layers 412 may be described as “dense” because each of the neurons in a given layer may receive an input from each neuron in a previous layer and provide an output to each neuron in a subsequent layer. The dense layers 412 may be followed by an output layer 408 that provides the output of the convolutional neural network. The output may be, for example, an indication of which class, from a set of classes, the image 402 (or any portion of the image 402) belongs to.

It should be appreciated that the convolutional neural network shown in FIG. 4 is only one example implementation and that other implementations may be employed. For example, one or more layers may be added to or removed from the convolutional neural network shown in FIG. 4. Additional example layers that may be added to the convolutional neural network include: a rectified linear units (ReLU) layer, a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output.

Convolutional neural networks may be employed to perform any of a variety of functions described herein. For example, a convolutional neural network may be employed to identify an anatomical location in an ultrasound image. For further discussion of deep learning techniques, see U.S. patent application Ser. No. 15/626,423 titled “AUTOMATIC IMAGE ACQUISITION FOR ASSISTING A USER TO OPERATE AN ULTRASOUND DEVICE,” filed on Jun. 19, 2017 and published as U.S. Pat. Publication No. 2017/0360401 A1 (and assigned to the assignee of the instant application), which is incorporated by reference herein in its entirety.

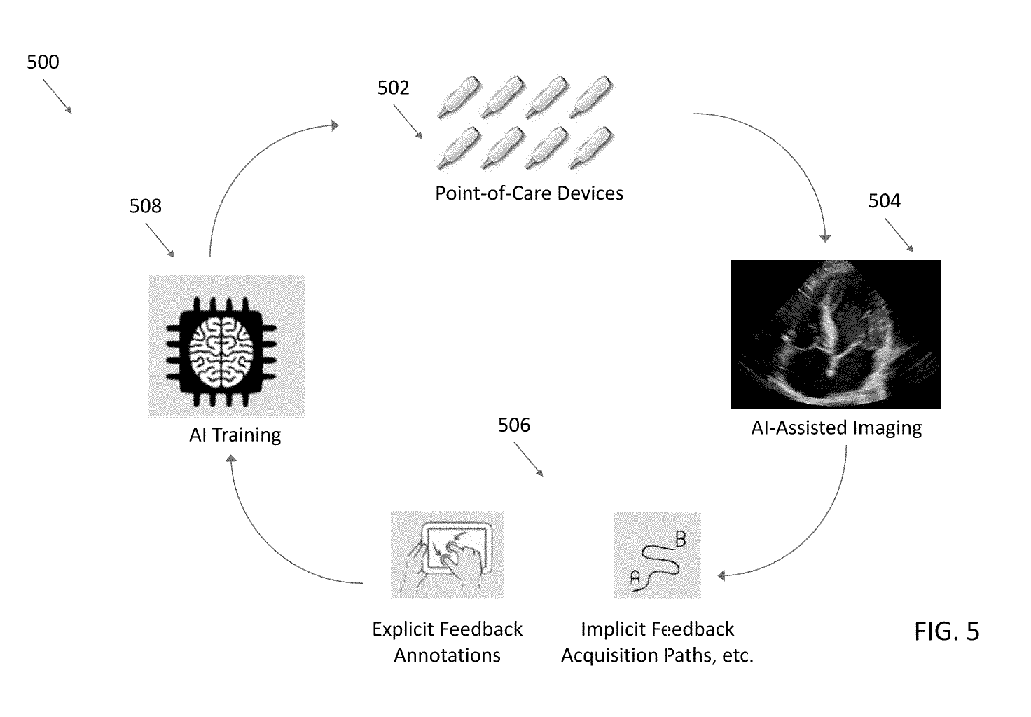

FIG. 5 shows an example of a “virtuous circle” 500 for continuously improving the performance of certain processes and systems described herein. Stage 502 shows point-of-care devices (e.g., any of the ultrasound devices discussed previously). Each of the point-of-care devices may be associated with a different individual, group of individuals, institution, or group of institutions, and may be in different locations where care using the plurality of point-of-care devices is administered, such as a patient's home, a medical clinic, a doctor's office, an ambulance, a hospital, etc. Stage 504 shows that each of the point-of-care devices shown in stage 502 may be used to perform artificial intelligence (AI)-assisted imaging. For example, each of the point-of-care devices may be used to perform FAST or eFAST exams in which anatomical locations being imaged are automatically identified using AI (e.g., using statistical models and/or deep learning techniques) and the identified anatomical location may be used to automatically select an ultrasound imaging preset corresponding to the anatomical location, as discussed previously. The AI-assisted imaging may produce images, such as ultrasound images. Stage 506 shows that the images produced in stage 504 may be uploaded to one or more servers (e.g., a “cloud” configuration), and from the cloud the images may be accessed by individuals or algorithms and processed to extract information that can be used as sources of feedback for statistical models (as shown in stage 508). For example, the images may be analyzed by annotators (e.g., sonographers or doctors) to identify anatomical locations in the images (explicit feedback). As another example, in cases in which FAST or eFAST exams image a specific set of anatomical locations in a specific sequence, a sequence of the images may be automatically processed by algorithms to identify anatomical locations in the images (implicit feedback). Stage 508 shows that the explicit and implicit feedback from stage 506 may be used to train statistical models hosted on the cloud to more accurately identify anatomical location in images, as discussed above in relation to FIG. 4. The trained models may be downloaded from the cloud to the point-of-care devices shown in stage 502 (e.g., across a wired or wireless communication link), and used to more accurately perform AI-assisted imaging as shown in stage 504. Accordingly, as more point-of-care devices are deployed, more AI-assisted imaging is performed, more images are produced and analyzed to extract data, and models are trained with more data extracted from images, the point-of-care devices and the AI-assisted imaging they perform may continue to improve as they are updated by the most recent AI training.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. A method, comprising:
configuring, with a processing device in operative communication with an ultrasound device, the ultrasound device with different sets of imaging parameter values without intervening imaging parameter configuration user input.

2. The method of claim 1, wherein configuring the ultrasound device with the different sets of imaging parameter values is based at least in part on determining that the ultrasound device has changed from imaging a first anatomical location to imaging a second anatomical location.

3. The method of claim 2, wherein determining that the ultrasound device has changed from imaging the first anatomical location to imaging the second anatomical location is performed by analyzing ultrasound data collected by the ultrasound device or by analyzing at least one ultrasound image generated based on the ultrasound data collected by the ultrasound device.

4. The method of claim 2, wherein determining that the ultrasound device has changed from imaging the first anatomical location to imaging the second anatomical location is performed by analyzing an optical image or an optical video obtained with a camera on the processing device.

5. The method of claim 2, wherein determining that the ultrasound device has changed from imaging the first anatomical location to imaging the second anatomical location is performed by analyzing accelerometer data from the ultrasound device.

6. The method of claim 2, wherein determining that the ultrasound device has changed from imaging the first anatomical location to imaging the second anatomical location comprises using at least one statistical model.

7. The method of claim 1, wherein configuring the ultrasound device with the different sets of imaging parameter values is performed subsequent to and based on receiving a user selection of a FAST or eFAST exam preset.

8. A method, comprising:
automatically configuring, with a processing device in operative communication with an ultrasound device, the ultrasound device with a set of imaging parameter values based on automatically determining an anatomical location being imaged by the ultrasound device.

9. The method of claim 8, wherein determining the anatomical location being imaged by the ultrasound device comprises analyzing ultrasound data collected by the ultrasound device or analyzing at least one ultrasound image generated based on the ultrasound data collected by the ultrasound device.

10. The method of claim 8, wherein determining the anatomical location being imaged by the ultrasound device comprises analyzing an optical image or an optical video obtained with a camera on the processing device.

11. The method of claim 8, wherein determining the anatomical location being imaged by the ultrasound device comprises analyzing accelerometer data from the ultrasound device.

12. The method of claim 8, wherein determining the anatomical location being imaged by the ultrasound device comprises using at least one statistical model.

13. The method of claim 8, wherein automatically configuring the ultrasound device with the set of imaging parameter values based on automatically determining the anatomical location being imaged by the ultrasound device is performed subsequent to and based on receiving a user selection of a FAST or eFAST exam preset.

14. The method of claim 8, wherein automatically configuring the ultrasound device with the set of imaging parameter values based on automatically determining the anatomical location being imaged by the ultrasound device is performed without receiving user input regarding configuration of the ultrasound device.

15. The method of claim 8, wherein the set of imaging parameter values corresponds to the anatomical location being imaged by the ultrasound device.

16. A method of operating an ultrasound device for a FAST or eFAST exam, comprising:
automatically configuring, with a processing device in operative communication with the ultrasound device, the ultrasound device with a cardiac preset when a cardiac region is being imaged and automatically configuring the ultrasound device with an abdominal preset when an abdominal region is being imaged during the FAST or eFAST exam.

17. The method of claim 16, further comprising:
automatically determining whether the cardiac region or the abdominal region is being imaged by the ultrasound device.

18. The method of claim 17, wherein determining whether the cardiac region or the abdominal region is being imaged by the ultrasound device comprises analyzing ultrasound data collected by the ultrasound device or analyzing at least one ultrasound image generated based on the ultrasound data collected by the ultrasound device.

19. The method of claim 17, wherein determining whether the cardiac region or the abdominal region is being imaged by the ultrasound device comprises analyzing an optical image or an optical video obtained with a camera on the processing device.

20. The method of claim 17, wherein determining whether the cardiac region or the abdominal region is being imaged by the ultrasound device comprises analyzing accelerometer data from the ultrasound device.

21. The method of claim 17, wherein determining whether the cardiac region or the abdominal region is being imaged by the ultrasound device comprises using at least one statistical model.

22. The method of claim 16, wherein automatically configuring the ultrasound device with a cardiac preset when a cardiac region is being imaged and automatically configuring the ultrasound device with an abdominal preset when an abdominal region is being imaged during the FAST or eFAST exam is performed without receiving user input regarding configuration of the ultrasound device.

23. An apparatus, comprising:
a processing device in operative communication with an ultrasound device and configured to:
configure the ultrasound device with different sets of imaging parameter values without intervening imaging parameter configuration user input.

24. The apparatus of claim 23, wherein the processing device is configured to configure the ultrasound device with the different sets of imaging parameter values based at least in part on determining that the ultrasound device has changed from imaging a first anatomical location to imaging a second anatomical location.

25. The apparatus of claim 24, wherein the processing device is configured to determine that the ultrasound device has changed from imaging the first anatomical location to imaging the second anatomical location by analyzing ultrasound data collected by the ultrasound device or by analyzing at least one ultrasound image generated based on the ultrasound data collected by the ultrasound device.

26. The apparatus of claim 24, wherein the processing device is configured to determine that the ultrasound device has changed from imaging the first anatomical location to imaging the second anatomical location by analyzing an optical image or an optical video obtained with a camera on the processing device.

27. The apparatus of claim 24, wherein the processing device is configured to determine that the ultrasound device has changed from imaging the first anatomical location to imaging the second anatomical location by analyzing accelerometer data from the ultrasound device.

28. The apparatus of claim 24, wherein the processing device is configured to determine that the ultrasound device has changed from imaging the first anatomical location to imaging the second anatomical location using at least one statistical model.

29. The apparatus of claim 23, wherein the processing device is configured to configure the ultrasound device with the different sets of imaging parameter values subsequent to and based on receiving a user selection of a FAST or eFAST exam preset.

30. An apparatus, comprising:
a processing device in operative communication with an ultrasound device and configured to:
automatically configure the ultrasound device with a set of imaging parameter values based on automatically determining an anatomical location being imaged by the ultrasound device.

31. The apparatus of claim 30, wherein the processing device is configured to determine the anatomical location being imaged by the ultrasound device by analyzing ultrasound data collected by the ultrasound device or analyzing at least one ultrasound image generated based on the ultrasound data collected by the ultrasound device.

32. The apparatus of claim 30, wherein the processing device is configured to determine the anatomical location being imaged by the ultrasound device by analyzing an optical image or an optical video obtained with a camera on the processing device.

33. The apparatus of claim 30, wherein the processing device is configured to determine the anatomical location being imaged by the ultrasound device by analyzing accelerometer data from the ultrasound device.

34. The apparatus of claim 30, wherein the processing device is configured to determine the anatomical location being imaged by the ultrasound device using at least one statistical model.

35. The apparatus of claim 30, wherein the processing device is configured to automatically configure the ultrasound device with the set of imaging parameter values based on automatically determining the anatomical location being imaged by the ultrasound device subsequent to and based on receiving a user selection of a FAST or eFAST exam preset.

36. The apparatus of claim 30, wherein the processing device is configured to automatically configure the ultrasound device with the set of imaging parameter values based on automatically determining the anatomical location being imaged by the ultrasound device without receiving user input regarding configuration of the ultrasound device.

37. The apparatus of claim 30, wherein the set of imaging parameter values corresponds to the anatomical location being imaged by the ultrasound device.

38. An apparatus configured to operate an ultrasound device for a FAST or eFAST exam, comprising:
a processing device in operative communication with the ultrasound device and configured to:
automatically configure the ultrasound device with a cardiac preset when a cardiac region is being imaged and automatically configure the ultrasound device with an abdominal preset when an abdominal region is being imaged during the FAST or eFAST exam.

39. The apparatus of claim 38, wherein the processing device is further configured to automatically determine whether the cardiac region or the abdominal region is being imaged by the ultrasound device.

40. The apparatus of claim 39, wherein the processing device is configured to determine whether the cardiac region or the abdominal region is being imaged by the ultrasound device by analyzing ultrasound data collected by the ultrasound device or analyzing at least one ultrasound image generated based on the ultrasound data collected by the ultrasound device.

41. The apparatus of claim 39, wherein the processing device is configured to determine whether the cardiac region or the abdominal region is being imaged by the ultrasound device by analyzing an optical image or an optical video obtained with a camera on the processing device.

42. The apparatus of claim 39, wherein the processing device is configured to determine whether the cardiac region or the abdominal region is being imaged by the ultrasound device by analyzing accelerometer data from the ultrasound device.

43. The apparatus of claim 39, wherein the processing device is configured to determine whether the cardiac region or the abdominal region is being imaged by the ultrasound device using at least one statistical model.

44. The apparatus of claim 38, wherein the processing device is configured to automatically configure the ultrasound device with a cardiac preset when a cardiac region is being imaged and automatically configuring the ultrasound device with an abdominal preset when an abdominal region is being imaged during the FAST or eFAST exam without receiving user input regarding configuration of the ultrasound device.
